(19) United States
(12) Patent Application Publication
(10) Pub. No.: US 2016/0095710 A1
(43) Pub. Date: Apr. 7, 2016

(54) IMPLANTS COMPRISING ANCHORING ELEMENTS

(71) Applicant: CERAMTEC GMBH, Plochingen (DE)

(72) Inventors: Mateusz Juszczyk, Velden (DE); Alfons Kelnberger, Röthenbach (DE); Tina Mirus, Esslingen (DE); Heinrich Wecker, Eckental (DE); Frank Ziermann, Berlin (DE)

(21) Appl. No.: 14/889,712

(22) PCT Filed: May 7, 2014
(86) PCT No.: PCT/EP2014/059369

§ 371 Date: Nov. 6, 2015

(30) Foreign Application Priority Data
May 7, 2013 (DE) 10 2013 208 376.9

(51) Int. Cl. A61F 2/44

(52) U.S. Cl. CPC A61F 2/442; A61F 2220/0008; A61F 2310/00389; A61F 2310/0097

(57) ABSTRACT

Implants that have anchoring elements, which are vertebral implants that can be used as intervertebral disk replacement in the form of cages for the fusion of vertebral bodies.

The invention relates to implants comprising anchoring elements. The invention relates in particular to vertebral implants that can be used as intervertebral disk replacement in the form of cages for the fusion of vertebral bodies.

Endoprosthetic components for the fusion of vertebral bodies are well known. They are adapted in terms of their geometry to the anatomy of the human vertebral body, are located between two vertebral bodies, and replace the intervertebral disk completely or partially.

In a first phase of remaining in the human body, said endoprosthetic components typically keep the vertebral bodies spaced apart from one another and thus in an anatomically correct position solely by means of the mechanical properties of said endoprosthetic components. In a second phase, they facilitate the fusion and thus the adhesion of the two vertebral bodies surrounding the endoprosthetic components.

Known components for the fusion of vertebral bodies are based on metallic materials such as tantalum or titanium, on plastics such as highly cross-linked PE materials (polyethylene) or PEEK (polyetheretherketone), or on silicon nitride.

Metallic materials have the following disadvantages, for example:
Metallic abrasion and resulting negative effects on the human organism, e.g., foreign body reactions such as inflammatory or immunological reactions, tissue toxicity.
Artifacts and/or lack of translucency in imaging in medical diagnostics.
Aging effects and long-term behavior (fatigue, corrosion, release of metallic ions which can have toxic effects).

Components based on plastics such as highly cross-linked PE materials or PEEK can have the following disadvantages:
Insufficient mechanical properties such as the breaking off of teeth or other parts of the component, for example during fitting. This can have negative effects on the human organism.
Insufficient imageability in the common imaging methods (MRI, x-ray). Consequently, the use of metallic markers is required.
Aging effects and long-term behavior, in particular fatigue of material.

Also known are ceramic components, for example based on silicon nitride.

However, this class of material has been developed with a view on excellent high-temperature properties—for example for mechanical processing of metallic components for the automotive industry—and, compared with other ceramic high-performance materials based on oxidic systems, is rather ranked in the middle in terms of properties required for the use as a medical implant, such as strength, hardness, and long-term stability.

Moreover, this involves a material composed of a plurality of components with needle-shaped silicon nitride particles embedded in a glass matrix. Sintering of the material is accordingly complicated. Also, as a result of this, mechanical processing such as grinding or polishing is extremely challenging and difficult.

moreover, components made from Si₃N₄ exhibit gray to black coloring which, for purely visual and esthetic reasons, are poorly accepted in the medical field.

All these disadvantages increase the production costs of the components, which constitutes another disadvantage.

A fundamental problem, which increasingly becomes the focus in the case of implantation surgery, is the risk of infection during surgery. This risk can be reduced with ceramic components, the surface properties of which can have an inhibitory effect on bacterial colonization, for example. Thus, is desirable to have improved ceramic implants available, in particular for use in the spinal region.

Known ceramic cages usually are ring-shaped and/or adapted to the shape of the human vertebral body, wherein the ring is composed of a monolithic, thus dense, strong and very stiff ceramic material. In the center, these cages have a hollow space which either is filled with known bone replacement materials (autologous, allogeneic or synthetic) or has an artificial porous osseoinductive or osseoconductive structure. Usually, the osseoconductive or osseoinductive structure is less stiff than the outer ring. In this region, bone cells should build up new bone material, wherein the cells involved in this need a corresponding mechanical stimulus.

Due to the relatively complex biomechanics of the spine, very different load states can occur, which can be expressed in the form of micro-mechanical movements or, caused by flexion or extension of the spline, in the form of macro-mechanical movements of the section to be fused.

Both forms of movement should be avoided during the fusion since they can, of course, also affect the position of the implant and the healing process.

In principle, there are various approaches to avoid the macro-movements and accordingly different configurations of the implants.

The cages can be fixed anteriorly or posteriorly by means of a separate pedicle screw system or by means of a plate-and-screw system in that the adjacent vertebral bodies are connected to one another in an angularly stable manner so that, for example during flexion of the spine, the vertebral bodies and the implant do not move with respect to one another and do not separate undesirably. Also, so-called “stand alone” cages are used, which additionally have end plates with receptacles or integrated receptacles for screws for the direct fixation of the implant in the vertebral bodies.

The additional screw connection of the segment to be fused has also the effect that the two vertebral bodies and the implant rest on top of one another under a certain pressing force, which can facilitate the healing process and supports the desired freedom from pain.

In order to avoid micro-movements, cages have teeth-shaped structures on the upper side and/or lower side of the implant, which engage by anchoring in the end plates of the vertebral bodies and thus provide for a certain stability of the implant, the so-called primary stability.

However, these anchoring elements can result in damage to or even destruction of the end plates of the vertebral bodies during fitting, as a result of which a fusion can take place only to a limited extent or not at all or the cages sink into the end plates of the vertebral bodies and thus negatively influence the repositioning result.

The following problem exists: If the anchoring element is too weak, it does not damage the end plates, but it does not provide for sufficiently high primary stability. If the anchoring is too strong, it damages the end plates during fitting, resulting in the above-mentioned negative consequences.

It is an object of the invention to provide an implant which, in particular, is suitable for the fusion of vertebral bodies. The implant should be provided with anchoring elements which enable safe and low-damage implantation and, at the same time, allow secure anchoring between the vertebral bodies. The implant should preferably be composed of ceramics.

Such an implant should meet the following requirements:
The implant should not sustainably damage or destroy the end plates of the vertebral bodies during the implantation, since this can entail negative effects on a successful fusion and/or on the mechanical integrity of the vertebral bodies.
During the implantation, the implant should not be damaged by mechanical loads in such a manner that the implant is fractured due to subsequent biomechanical loads.
After the implantation, the implant should ensure sufficiently high primary stability. In other words, during the fusion, the implant must remain between the vertebral bodies in a stable manner and must not change its position due to biomechanical load.
The implant must not be damaged by biomechanical load during the fusion in such a manner that the implant is fractured.
The implant should ensure successful stabilization of the damaged spinal segment and should ensure a high fusion rate of the two vertebral bodies.

The object of the invention is achieved by a ceramic vertebral implant having the features according to claim 1.

An implant according to the invention has an upper side, a lower side and a shell surface, wherein the shell surface can be subdivided into front, rear and side surfaces, if necessary. The upper and/or lower side have/has anchoring elements for connection to adjacent osseous skeleton elements.

Preferably, the implant is a vertebral implant. More preferably, it is a cage for the fusion of vertebral bodies, wherein the anchoring elements serve for connection to the end plates of adjacent vertebral bodies.

According to a preferred embodiment of the invention, the implant is composed of a ceramic material. Particularly preferred are oxide ceramics, in particular from the class of aluminum oxides or zirconium oxides or from mixtures of both. Particularly advantageous are extremely damage-tolerant materials such as dispersoid ceramics stabilized with rare earths, in particular Gd and/or Sa. The dispersoid ceramic material is preferably composed of zirconium oxide, more preferably comprising percentages of aluminate.

According to another preferred embodiment of the invention, the vertebral implants can have a bioactive coating. A bioactive coating is to be understood as a coating that establishes a connection with the adjacent bone. Such coatings can in particular be composed of or comprise hydroxyapatite and/or tricalcium phosphate. However, coatings based on bioglasses are also suitable. Other bioactive coatings, i.e., for example, coatings acting in an osseoconductive and/or osseoinductive manner, are also possible. Coatings having an antimicrobial effect are also conceivable.

The coating of the components serves for bioactivation, which ensures that bone-forming cells adhere well, are provided with cytocompatible conditions, and become osteogenetically active.

According to a particularly preferred embodiment of the invention, the vertebral implants can have a central cavity that extends at least through the upper and/or lower side so that bone regeneration of the adjacent vertebral bodies can take place through the implant. Osseoconductive and/or osseoinductive materials (autologous, allogenic, artificial) which support bone growth and/or provide a suitable scaffold for ingrowth of bone cells and/or support the vascularization of the newly formed tissue can advantageously be introduced into such cavities.

In order to ensure good and sustainable fixation of the vertebral implant, the shell surface of the implant can be provided with at least one opening, through which the implant can be screwed to at least one adjacent vertebral body. The opening should be designed in a ceramic-appropriate manner, i.e., for example, sharp edges are to be avoided so as to reliably prevent point loads when in contact with a screw. Point loads acting on ceramics can result in failure of the entire component due to fracture and therefore should principally be avoided.

The screws can be composed of metals or metal alloys which are commonly used in implantation technology. Particularly preferably, the screws can also be composed of a ceramic-comprising material. Particularly preferred here is a zirconium-oxide-comprising material, for example an ATZ (alumina-toughened zirconia) ceramic. However, the screws could also be composed of PEEK or polymer material. This would have the advantage that the problem of point loads on the ceramic is significantly reduced. Moreover, all these materials have the advantage that they do not result in artifacts during imaging examinations and do not negatively influence imageability.

In order to achieve an optimal fit of the vertebral implant in the intervertebral space, the upper and/or lower sides can be convexly curved in the longitudinal direction and/or transverse direction so that they fill the intervertebral disk space to the largest possible extent and with as precise a fit as possible.

According to another preferred embodiment, the upper and lower sides can be arranged substantially plane-parallel to one another. In order to be able to restore the lordosis or kyphosis of a healthy spine, it can also be of advantage if the upper and lower sides of the vertebral implants are arranged at an angle to one another.

In order to ensure secure anchoring of the implant in the intervertebral space, a preferred embodiment provides that at least the entire upper and/or lower side of the implant is structured by anchoring elements. Of course, by appropriately designing the implant, it is also possible that only subsections of the top side and/or bottom side are provided with anchoring elements.

The longitudinal extent of the anchoring elements can be arranged perpendicular to the implantation direction so that an area as large as possible is available, which holds the implant in its place counter to the implantation direction. If, in addition, the anchoring elements are arranged in parallel rows, movement in the channel created by inserting the implant can be effectively prevented.

According to another preferred embodiment of the invention, the anchoring elements can also extend in curves so that areas as large as possible of the upper and/or lower side of the implant can be provided with anchoring elements. This arrangement of the anchoring elements has the advantage that first subsections of the anchoring elements prevent the implant from slipping counter to the implantation direction and second subsections of the anchoring elements prevent slipping perpendicular to the implantation direction. Thus, slipping of the implant forwards or backwards or to the right or the left in the intervertebral disk space can be effectively avoided.

The curved anchoring elements can be arranged concentrically.

The anchoring elements can also be arranged semicircularly and/or can comprise portions of semicircular curves. A sequence of differently aligned curves results in a serpentine-like arrangement which is also subsumed under the term “curves”. Such arrangements of the anchoring elements have the same effect as the previously described embodiment.

A rib structure having a shark-fin-like cross-section, i.e., ribs having a longer flank and an opposite shorter flank, has proven to be a particularly advantageous shape for the anchoring elements. The shorter flank has a steeper angle so that the flank can serve as a counter bearing against slipping. However, in the direction of the long flank, insertion into an intervertebral disk space is possible without any problem.

Another embodiment of a vertebral implant according to the invention is composed of at least two components that can be plugged together, a first and a second component. Such an implant has a first and a second outer contour. The first outer contour requires more space than the second outer contour and, according to a particularly preferred embodiment of the invention, is formed by the components when the components are plugged together only partially or incompletely. For implantation, the anchoring elements of the first component are received in the recesses of the second or a further component, and the anchoring elements of the second component are received in the recesses of the first or a further component in such a manner that the first outer contour of the vertebral implant is substantially smooth.

In this state, the components can advantageously be kept apart from one another, for example by spacers or advantageously by the implantation instrument, so that the anchoring elements do not protrude beyond the other surfaces of the implant, in particular the upper and/or the lower side.

This has the advantage that the anchoring elements cannot get caught in the tissue during implantation and/or cannot damage the tissue. The component having the first outer contour has a greater height than the component having the second outer contour; however, it still can be inserted into an intervertebral disk space without problems and without causing injuries.

The implant having the second outer contour requires less space than the component having the first outer contour and corresponds to the implant after the implantation, that is to say, in the “functional state”. The outer contour is characterized in that the anchoring elements protrude beyond the other surfaces of the implant, in particular the upper and/or the lower side, and can form a slip-resistant connection with the end plates of the adjacent vertebral bodies. According to preferred embodiments of this implant, the smaller volume is obtained by pushing together the components, in particular in the vertical direction. Pushing together or displacing the components in the context of this invention is to be understood as horizontal, vertical or transversal displacement. However, the turning of one of the components shall not be comprised by this term.

Known from the prior art are expandable cages made from metal which provide solutions in which, after implantation, points provided with cutting edges cut into the end plates of adjacent vertebral bodies by rotating about a horizontal axis.

However, the solution known from the prior art cannot be implemented by means of a ceramic component, because relatively delicate parts such as the anchoring elements composed of tips with cutting edges would be subjected during the rotation to high loads which are not suitable for ceramics. It can be expected that the ceramic tips would not be able to or would only insufficiently be able to withstand the bending and tensile load and that component failure could result.

In contrast, the solution comprising “extendable” tips or anchoring elements as proposed herein is a ceramic-appropriate solution which eliminates bending and tensile loads of the implant. The proposed solution results substantially in compression loads which can easily be absorbed by ceramic components.

Another advantage of the solution described here is that the end plates of the adjacent vertebral bodies are injured no more than necessary on the way into the end position of the implant. The solution known from the prior art cuts through the end plates of the vertebral bodies in order to bring the tips of the anchoring elements into their final position. The solution described herein moves the anchoring elements into the end plate substantially perpendicularly to the surface of the end plate so that injuries can only occur at the entry points.

In the solution according to the prior art, the marks resulting from the cutting of the anchoring elements represent weak points with respect to the anchoring since no optimal counter bearing for the anchoring elements is available in this direction. The solutions presented herein avoid this disadvantage as well.

According to a particularly preferred embodiment of the invention, the first and the second components are of identical shape. The components can then be plugged together in such a manner that the upper side of the first component is arranged on the upper side of the second component. This embodiment has the advantage that only one component has to be produced for the entire implant, which is of interest particularly from an economic point of view.

The anchoring elements of this embodiment can be spike-like projections or tips. This shape is particularly suitable, because it can be inserted into receptacles of another component without any problem and, at the same time, provides good support on the end plates of the adjacent vertebral bodies.

In another embodiment, the anchoring elements can be triangular projections which can be received in triangular recesses of the other component by insertion.

Basically, all shapes that can be transferred by moving, in particular in the vertical direction, from a position in which the shape is received in the implant into a position in which the shape protrudes beyond the implant are possible for such anchoring elements.

If not only an intervertebral disk is to be replaced by the vertebral implant, but rather, for example, a whole vertebral body, it is also possible to arrange a further component between the first and the second components, according to the modular design principle. In this case, the further component has to provide the anchoring elements which are received in the recesses of the first and/or second component for implantation.

Moreover, the implant advantageously has structures with which an instrument is in secure engagement for implantation.

The above-described vertebral implants can be used as intervertebral disk implants and in particular as cages for the fusion of adjacent vertebral bodies.

The components of the vertebral implants can be molded as pressed components in the green state or can be injection molded in large quantities by means of ceramic injection molding methods. Subsequently, the components are treated thermally, i.e., sintered, optionally hot-isostatically pressed, white-fired, and the surfaces are mechanically finished, for example ground or polished, if necessary.

The invention is described in more detail below with reference to the accompanying drawings. In the figures:

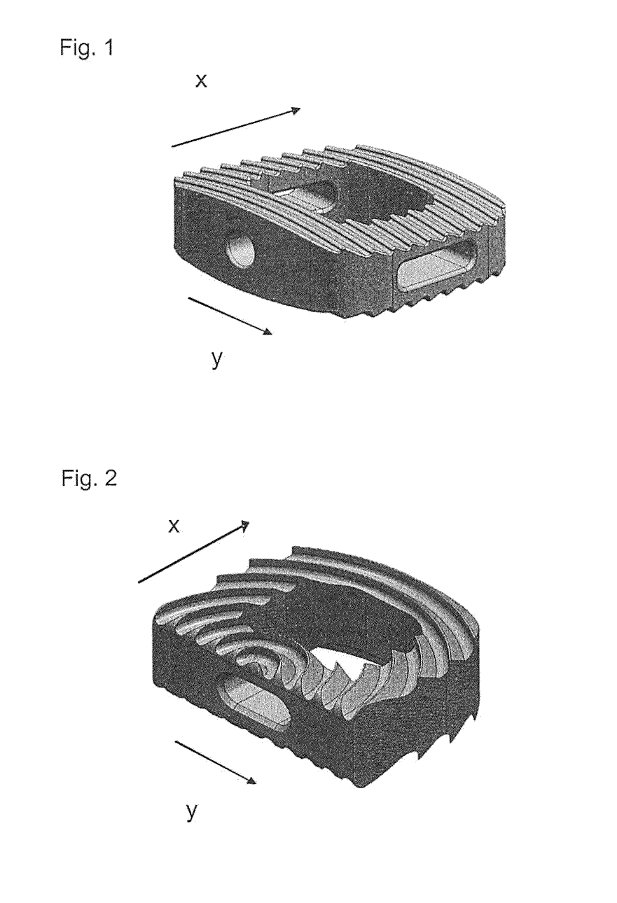

FIG. 1 shows a vertebral implant having a parallel rib structure;

FIG. 2 shows a vertebral implant having a concentric rib structure;

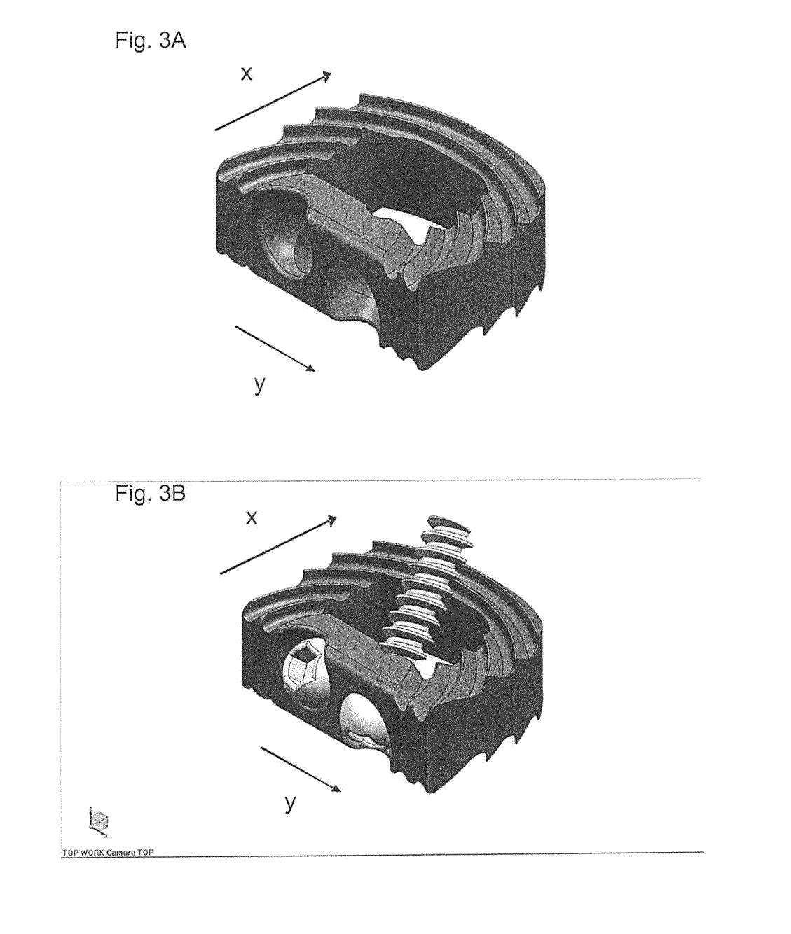

FIG. 3A shows a vertebral implant having screw receptacles;

FIG. 3B shows a vertebral implant as in FIG. 3A having screws;

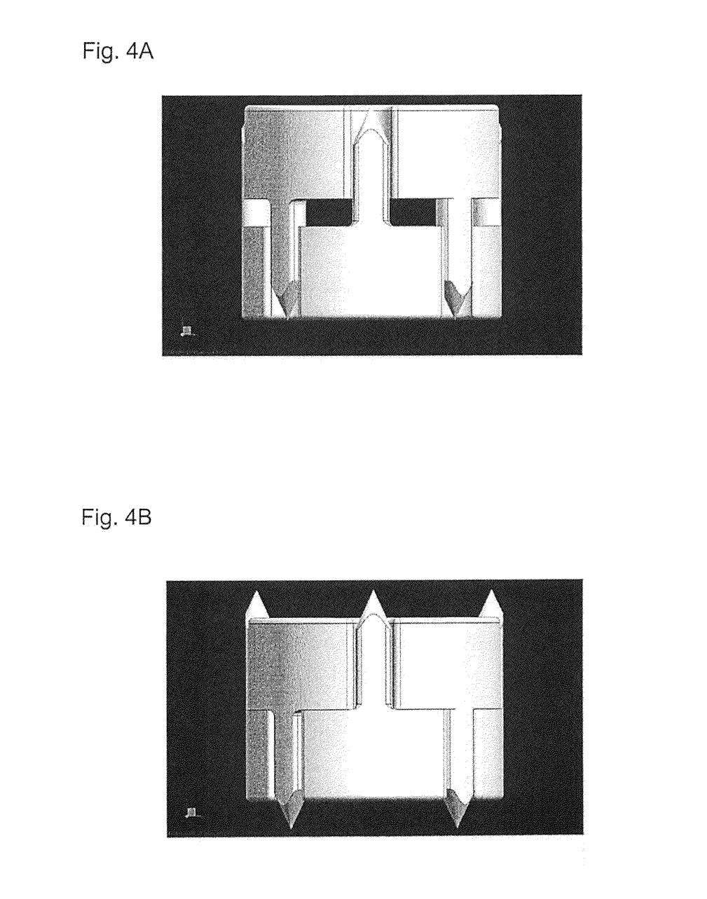

FIG. 4A shows a vertebral implant composed of two components having spike-shaped anchoring elements and a first outer contour suitable for implantation;

FIG. 4B shows the vertebral implant of FIG. 4A having a second outer contour in the implanted state;

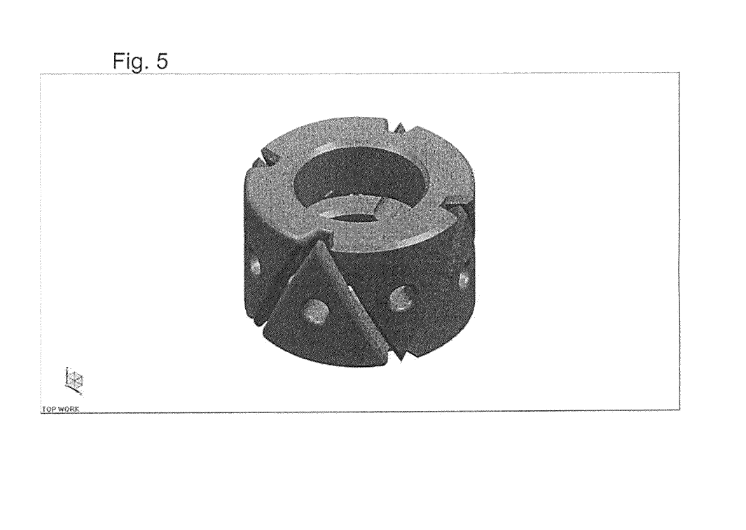

FIG. 5 shows a vertebral implant composed of two components having triangular anchoring elements.

FIG. 1 shows a vertebral implant in the form of a cage for fusing adjacent vertebral bodies, having anchoring elements in the form of a parallel rib structure.

The cage is composed of an Al₂O₃ material reinforced with zirconium oxide and having a high hardness and bending strength, which has established itself as a biocompatible material in medical technology.

The upper and lower sides of the vertebral implant are adapted to the anatomy of the end plates of the vertebral bodies and have in each case a convex surface in the x- and y-directions.

The upper and lower sides of the implant can be arranged plane-parallel to one another or can be arranged at an angle to one another (lordosis). This embodiment has a lordosis angle of 7° and therefore takes account for patient-specific anatomical requirements.

The anchoring elements, here teeth or ribs on the upper and lower sides of the implant, extend over the entire surface and are arranged parallel to one another. Through their shark-fin-like structure, they enable a preferably anterior implantation in the x-direction and prevent micro-movement in the opposite direction.

The radii of the teeth are shaped such that, on the one hand, they satisfy the mechanical requirements of the material and the forming-related possibilities of the material and, on the other, also enable maximum hold and primary stability of the component.

If a material having higher toughness and damage tolerance, for example a zirconium-oxide-based material, is used, these tooth structures can be shaped even more distinctly and with smaller radii so that an even higher primary stability is achieved.

The circular recess in the front shell surface enables the insertion of an instrument for the secure implantation of the component.

At the same time, this recess can also be utilized to introduce bone-forming materials into the interior of the cage, for example in the form of an injectable cement based on hydroxyapatite or tricalcium phosphate.

The two oval recesses in the lateral shell surfaces have the advantage that newly formed bone material can accumulate and grow therein, which results in additional stabilization of the component and the fused vertebral bodies.

The geometry is selected such that a certain critical distance, which can no longer be bridged by the bone cells, is not exceeded (critical size bone defect).

FIG. 2 shows a vertebral implant having anchoring elements in the form of a concentric rib structure.

This embodiment of a cervical cage is made from the same ceramic material as the preceding exemplary embodiment, namely from an Al₂O₃ ceramic reinforced with zirconium oxide. It has a different tooth or rib structure, wherein the ribs are arranged concentrically to one another in principle and have different radii.

This structure too enables the already described low-damage implantation in the one direction, but it prevents not only micro-movement in the one direction but also movement in the y-direction, thus perpendicular to the implantation direction.

An advantage is primary stability that is increased in comparison to the previously described embodiment.

Also, the tooth structure can look differently, in principle; what is important is only that it ensures additional stabilization in the y-direction.

In addition to the above-described embodiment, the cage shown can have two recesses which are located at the front anterior shell surface, see FIG. 3A, and which can receive screws, which can be screwed into the vertebral bodies for additional fixation. The same cage having inserted screws in shown in FIG. 3B.

The screws are advantageously made from a zirconium-oxide-based material, because, in particular in view of toughness and damage tolerance of this material, suitable screw structures adapted to the vertebral body bone can be implemented therewith.

Particular attention is to be paid to the fact that point contact is avoided when the screw heads contact the two recesses because this can lead to high local stresses at these contact surfaces, which consequently can result in failure of the ceramic material due to fracture.

Furthermore, a notch effect occurs at sharp ceramic component edges, which are often created in the case of countersunk screws. These edges can be starting points for cracks which, at least in the medium term, can result in failure of the component.

This point contact can be avoided, for example, by means of a particularly ceramic-appropriate design of the recesses or by means of an insert component composed of a suitable material, such as plastics, which can securely absorb the point loads.

FIGS. 4A and 4B show a vertebral implant that is composed of two components having spike-shaped anchoring elements. This vertebral implant can likewise be used as a cage for the fusion of vertebral bodies.

Thus, this is a cervical cage that requires no additional fixation for avoiding macro-movements.

In a particularly preferred embodiment, the implant according to the invention comprises two identical components. The two components in combination with one another form the implant according to the invention and are connected to one another in a positive locking and movable manner.

These multi-part cages are likewise preferably composed of ceramics, but, of course, they can also be composed of other common implant materials, such as PEEK or titanium or titanium alloys.

In a first state of the combination, the spike-shaped anchoring elements are not effective, i.e., they do not protrude beyond the respective surfaces (here: upper and lower sides of the implant). This represents the state during the implantation. The implant has the first outer contour according to the definition.

In a second state of the combination, the two components lie flat on top of one another and the spike-shaped anchoring elements protrude beyond the respective surfaces. This represents the state after the implantation, see FIG. 4B. The implant has the second outer contour according to the definition.

This mechanism enables a smooth, damage-free implantation and secure anchoring by means of anchoring elements which extend automatically after the implantation.

When the instrument used for inserting the implant into the intervertebral disk space is removed, the implant anchors itself independently and automatically in the desired position due to the self-extracting teeth that are under load.

Moreover, the implant has structures with which an instrument is in secure engagement for implantation and by means of which a spreading and release according to the invention for self-extraction are possible.

A particular advantage of this embodiment is that, in the case of a spreading of the two components by a distance of x mm, the implant can release spike-shaped anchoring elements that have a length of two times x mm.

A second ceramic-appropriate variant that is based on the same principle as the embodiment in FIG. 4 is shown in FIG. 5. However, triangular anchoring elements are provided here instead of the spike-shaped anchoring elements.

Moreover, the implant has circular holes or recesses that can be used for receiving an instrument for implantation. If these holes are configured adequately, they can also be used for screws for fixing the component in adjacent vertebral bodies.

1.-17. (canceled)

18. A vertebral implant, comprising:
an upper side;
a lower side; and
a shell surface, wherein the shell surface is subdivided into front, rear and side surfaces; and
wherein the implant has anchoring elements for connection to end plates of adjacent vertebral bodies.

19. The vertebral implant according to claim 18, wherein the implant comprises a ceramic.

20. The vertebral implant according to claim 19, wherein the ceramic is an oxide ceramic material.

21. The vertebral implant of claim 18, wherein the ceramic is selected from the group consisting of an aluminum oxide and a zirconium oxide.

22. The vertebral implant according to claim 18, wherein the vertebral implant further comprises a bioactive coating.

23. The vertebral implant according to claim 22, wherein the bioactive coating comprises at least one member selected from the group consisting of hydroxyapatite, tricalcium phosphate and a bioglass.

24. The vertebral implant according to claim 18, wherein the implant has a central cavity that extends at least through the upper and/or lower side so that bone regeneration of the adjacent vertebral bodies can take place through the implant.

25. The vertebral implant according to claim 18, wherein the shell surface has at least one opening through which the implant can be screwed to at least one adjacent vertebral body.

26. The vertebral implant according to claim 25, wherein the screws are composed of a ceramic-comprising material, in particular a zirconium-oxide-comprising material.

27. The vertebral implant according to claim 18, wherein at least one of the upper side and the lower side is convexly curved in a longitudinal or a transverse direction.

28. The vertebral implant according to claim 18, wherein the upper and lower sides are arranged plane-parallel or at an angle to one another.

29. The vertebral implant according to claim 18, wherein a longitudinal extent of the anchoring elements runs perpendicularly to the implantation direction.

30. The vertebral implant according to claim 18, wherein the anchoring elements extend in curves so that first subsections of the anchoring elements prevent the implant from slipping counter to the implantation direction and second subsections of the anchoring elements prevent slipping perpendicular to the implantation direction.

31. The vertebral implant according to claim 30, wherein the anchoring elements are arranged semicircularly.

32. The vertebral implant according to claim 18, wherein the vertebral implant comprises at least one first component and one second component, wherein the first component and the second component s can be plugged together;
wherein the vertebral implant has a first outer contour for implantation and a second outer contour in the implanted state; and
wherein the anchoring elements are received in recesses of the first or second component for implantation.

33. The vertebral implant according to claim 32, wherein the anchoring elements are received in recesses of the first and/or the second component in such a manner that the first outer contour of the vertebral implant is substantially smooth, and by displacing the two components, the second outer contour is formed, in which case the anchoring elements protrude beyond an upper and/or a lower side of the vertebral implant.

34. The vertebral implant according to claim 32, wherein a volume of the first outer contour for implantation is greater than a volume of the second outer contour in the implanted state.

35. The vertebral implant according to claim 32, wherein the first and the second components are shaped identically.

36. The vertebral implant according to claim 32, wherein an upper side of the first component is arranged on an upper side of the second component.

37. The vertebral implant according to claim 30, wherein the anchoring elements comprise sections of semicircular curves.
